(19) United States
(12) Patent Application Publication
(10) Pub. No.: US 2026/0137356 A1
(43) Pub. Date: May 21, 2026

(54) CONE BEAM COMPUTED TOMOGRAPHY SYSTEMS, METHODS, AND DEVICES

(71) Applicant: Ehmet Health, Inc., Plymouth, MI (US)

(72) Inventor: Neal H. Clinthorne, Ann Arbor, MI (US)

(21) Appl. No.: 19/391,217

(22) Filed: Nov. 17, 2025

Related U.S. Application Data
(60) Provisional application No. 63/721,012, filed on Nov. 15, 2024.

(51) Int. Cl. A61B 6/40; A61B 6/00; A61B 6/03; A61B 6/04; A61B 6/42

(52) U.S. Cl. CPC A61B 6/4085; A61B 6/032; A61B 6/0407; A61B 6/4208; A61B 6/4266; A61B 6/4291; A61B 6/5205; A61B 6/5282

(57) ABSTRACT

The present disclosure provides a cone beam computed tomography system for proton therapy comprising an x-ray source configured to generate x-rays and positioned to rotate around a patient at a treatment isocenter, a detector assembly positioned opposite the x-ray source and configured to receive x-rays that have passed through the patient, a rotation mechanism configured to rotate the x-ray source and the detector assembly around the patient while maintaining their relative positions, a translation mechanism configured to translate at least one of the x-ray source, the detector assembly, or a patient couch during rotation to implement a wobbled scan orbit, and a scatter rejection grid attached to the detector assembly and focused on the x-ray source to reduce scattered x-rays. The translation mechanism distributes missing projection data across multiple radii and angles, enabling improved reconstruction quality through iterative reconstruction methods.

### PRIORITY

Any and all applications for which a foreign or domestic priority claim is identified in the Application Data Sheet as filed with the present application are hereby incorporated by reference under 37 C.F.R. 1.57. This application claims the benefit of priority to U.S. Provisional Application No. 63/721,012, filed Nov. 15, 2024, the contents of which are incorporated by reference in their entirety as if set forth fully herein.

### TECHNICAL FIELD

The present disclosure relates to systems and methods for radiation therapy, including proton therapy. In particular, some implementations relate to imaging for use in proton therapy or other radiation therapy, including improved systems and methods for cone beam computed tomography.

### BACKGROUND

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

Cone beam computed tomography (CBCT) has become a common imaging modality in radiation therapy for patient positioning and treatment verification. However, there are significant challenges with CBCT imaging that make positioning, treatment verification, or both challenging and prone to error. Accordingly, improved approaches are needed.

### BRIEF DESCRIPTION OF THE DRAWINGS

Detailed descriptions of implementations of the present invention will be described and explained through the use of the accompanying drawings.

FIGS. 1A and 1B illustrate a top-down view of an imaging apparatus according to some implementations.

FIGS. 2A and 2B illustrate example geometries of systems according to FIGS. 1A and 1B, respectively.

FIGS. 3A-3C illustrate example sinograms according to some implementations.

FIG. 4 illustrates a flowchart for a cone beam computed tomography imaging method according to some implementations.

FIG. 5 is a block diagram depicting an embodiment of a computer hardware system configured to run software for implementing one or more of the systems and methods described herein.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

Although several implementations, embodiments, examples, and illustrations are disclosed below, it will be understood by those of ordinary skill in the art that the scope of the present disclosure extends beyond the specifically disclosed implementations, embodiments, examples, and illustrations and includes other uses of the inventions and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of certain specific implementations. In addition, implementations can comprise several novel features, and no single feature is necessarily essential or solely responsible for its desirable attributes.

Cone beam computed tomography (CBCT) is a common imaging modality in radiation therapy and can be used for tasks such as patient positioning and treatment verification. CBCT systems utilize a cone-shaped X-ray beam and a large area detector that can rotate around the patient to acquire projections from different angles. The projection images can be reconstructed to generate three-dimensional volumetric images that can be used to evaluate or verify patient positioning before administering a treatment. In CBCT, a plurality of X-ray projection images can be obtained as an X-ray source and detector system co-rotate around the patient.

In proton therapy, accurate patient positioning and dose calculation can be challenging for a variety of reasons, such as the finite range of protons in tissue and their sensitivity to tissue density variation. Unlike photon-based radiation therapy, where small positioning errors or anatomical changes may have a limited impact on dose distribution, designing a proton therapy treatment plan can involve detailed knowledge of proton stopping power and range. This makes high-quality imaging data particularly important for proton therapy applications. As radiotherapy, and specifically proton therapy systems, become more sophisticated, there is an increasing need to use patient alignment scans to ascertain information regarding the treatment, such as accurate positioning and accurate estimates of incremental and cumulative radiation dose to the target tumor, organs at risk, or both.

Current CBCT systems used in radiation therapy settings face several limitations that affect their utility for dose calculations, treatment planning, and so forth. The field of view for many existing systems is limited and often insufficient to encompass the entire cross-section of a patient at the treatment position. For example, one some systems, the field of view is limited to approximately 25 cm at the treatment isocenter, limiting the information available for clinical decision-making. Additionally, the image quality of conventional CBCT systems is lacking, which can lead to errors in positioning, incorrect dose calculations, or otherwise poor treatment planning and administration. The image quality of current CBCT scans, while usable for patient alignment, still has errors and can lack detail that would be advantageous for treatment planning. This can be especially important for proton therapy, where high-quality estimates of electron density are important in estimating proton stopping power and thus determining where treatment will be concentrated. Various factors can affect the imaging, such as X-ray scatter, incomplete sampling geometries, and detector limitations.

X-ray scattering presents a substantial challenge for CBCT imaging and can be especially problematic at larger angles. Scattered radiation detected by the imaging system degrades image quality and leads to inaccurate Hounsfield Unit (HU) values, which are used for tissue density determination. Scatter for the large cone angles used in CBCT is significantly greater than for the small angles used in helical CT. The circular orbit often employed in CBCT scanning also presents limitations, as it may provide incomplete sampling of the imaging volume, leading to reconstruction artifacts and reduced accuracy in resulting images. The circular orbit commonly used in CBCT imaging is often incomplete, meaning that 3D reconstruction cannot be done perfectly even in the absence of noise or scatter, as certain information is missing.

For a complete dataset in cone beam imaging, the focal spot of the detector source must cross every plane transecting the object. This limitation is not met when using a circular orbit and collecting more than one slice. To reduce incomplete data artifacts, the patient couch can be translated in the z direction (e.g., assuming x and y are orthogonal to each other and to the z axis, and the x and y axes are transverse to the CT axis, and that x is horizontal and y is vertical). However, this does not fully resolve the missing data problem because the patient is nearly always longer than the field of view (the “long object problem”), but it can substantially reduce attenuation non-uniformity arising from incomplete data and can improve spatial resolution in the axial direction for certain structures (e.g., Defrise phantom-like structures).

These issues can be especially pronounced in proton therapy systems, where the treatment nozzle used to provide protons to a treatment area takes up physical space that can limit the movement of the x-ray source and detector(s). Accommodating proton delivery hardware can also limit the size, shape, etc., of imaging equipment, which can limit the field of view or otherwise impact imaging.

Imaging speeds can also present a challenge, as extended scan times can increase the likelihood of patient motion artifacts and may be incompatible with breath-holding techniques that could otherwise improve image quality. Accordingly, it can be significant to limit acquisition times to avoid or at least limit the introduction of patient-motion-driven imaging artifacts.

As radiation therapy, including proton therapy, has become more sophisticated, high-quality imaging is of increasing importance. Imaging information can be used for patient positioning, treatment planning, and so forth. For example, it can be significant to evaluate dosage levels to determine incremental and/or cumulative dose to the target tumor, to organs at risk (OARs), or both. Radiation dose can be calculated using a planning CT scan; however, patient morphology can change during the course of treatment (e.g., if a patient loses weight). In some cases, diagnostic-quality CT can be performed for each treatment session to assess dose and potentially replan the remaining treatment; however, this is often impractical and can involve entirely separate imaging equipment.

One limitation of CBCT in radiotherapy with photons or particles (such as protons) is the field of view transverse to the rotational axis of the CBCT system. The field of view may fail to encompass the entire cross-section of the patient. For example, some systems may have a field of view of approximately 25 cm at the treatment position or isocenter. While the entire patient cross-section can be imaged away from the isocenter, the size of the x-ray detectors commonly used (e.g., 43 cm×43 cm) in the “half-fan” method may provide images with significant artifacts. Images may be useful for patient alignment, but HU values can have significant errors.

Another limitation is X-ray scatter. Scatter for the large angles used in CBCT imaging may be greater than for the scattering effects observed in helical CT. Detected scatter can result in poor fidelity of HU values relative to diagnostic CT scans. Described herein are hardware-and software-based approaches for limited and correcting scatter.

A further limitation is the orbit used in CBCT. The circular orbit may be incomplete. Thus, even without noise or scatter, 3D reconstruction cannot be performed perfectly as there is missing information. This can also lead to inaccurate HU values and possibly inaccurate proton dose calculations.

The present disclosure provides several approaches that can be used alone or in combination to address these and other limitations. A CBCT system as described herein may use a larger X-ray detector system, which can be assembled by tiling existing flat-panel X-ray detectors. Alternatively or additionally, the CBCT system can use one or more curved-panel x-ray detectors. In some implementations, the CBCT utilizes one or more scatter rejection grids on each detector, which can help reduce the scatter signal. Further, software can be used to estimate scatter and compensate for detected scatter. In some implementations, the system uses a scan orbit that is more complete or that better addresses the problem of missing information, which can lead to improved reconstruction accuracy and higher quality images that are better suited to dose calculation and treatment planning in proton therapy applications.

The CBCT system can include several components to enable imaging while the patient remains at the treatment position or under conditions in which the patient can be reliably returned to the treatment position, as described in more detail herein. The CBCT system can include an X-ray source configured to generate X-rays for imaging. The X-ray source can be positioned to direct X-rays through a patient, or more generally, any object to be imaged. In many systems, the X-ray source is placed farther from the isocenter than the detectors in order to improve image quality.

A detector assembly can be positioned to receive X-rays that have passed through the patient (or other object). The detector assembly can include one or more X-ray detectors configured to capture projection images. In some implementations, the detector assembly includes an array of X-ray detectors arranged to provide a greater field of view as compared to some single-detector implementations. The x-ray detector(s) can be flat, curved, etc., in different implementations. The detector(s) can be positioned generally opposite the x-ray source, with the patient positioned between the x-ray source and the detector assembly. In an example configuration, the detectors can include two detectors, which can be, for example, 43 cm×43 cm flat-panel x-ray detectors. The detectors can have a gap of, for example, from about 5 mm to about 20 mm, for example, 16 mm.

A rotation mechanism can be configured to rotate the X-ray source and detector assembly around the patient. The rotation mechanism can enable the X-ray source and detector assembly to co-rotate around a rotational axis while maintaining their relative positions. In some implementations, the rotation mechanism is configured to perform rotational movement while avoiding collision with proton treatment equipment, such as a treatment nozzle. This can limit the range of motion as the X-ray detector may be positioned at a distance that would interfere with the treatment nozzle or other components of the treatment system. In some implementations, short-arc scans are used. In a short-arc scan, the imaging system can rotate by, for example, 180 degrees plus the fan angle (angle subtended by the edges of the detectors with the vertex at the x-ray source) so that only the detectors need to clear the treatment nozzle.

The components of the CBCT system can be arranged relative to the patient and proton treatment equipment to enable imaging at the treatment isocenter. The treatment isocenter can be the position where the patient receives proton therapy treatment. By enabling imaging at the treatment isocenter, the system can enable patient positioning verification and dose calculation without requiring patient movement from the treatment position. This can be advantageous as any patient movement can result in the proton therapy dose being concentrated in the wrong area, which can result in ineffective treatment or even harm to nearby tissue or organs. This can be especially important as newer techniques, such as FLASH therapy, become available. In FLASH therapy, very high dose rates are used, shortening the treatment time and repetition considerably, for example, treatment over one to five sessions, with treatment times being on the order of about one second. Given such high, concentrated treatment dosages, it is important to ensure that treatment is delivered to the correct target location, as there is a significant risk of doing damage to nearby tissue and organs if the treatment location is incorrect.

The overall architecture of a CBCT system as described herein can accommodate constraints improved by the proton treatment environment. For example, the rotation mechanism and detector assembly can be configured to clear proton treatment nozzles and other equipment during rotation. However, the X-ray source is typically placed farther from the isocenter than the detectors to improve the field of view and image quality, and thus may not be able to clear a treatment nozzle or other equipment, limiting the rotation of the CBCT system. In some implementations, the system rotates through arcs that form incomplete circles to avoid mechanical interference. However, this results in missing information as scan data cannot be acquired at all angles.

Another issue is that tiled detectors, while increasing the detector area, can also have dead spots or gaps, such as at the intersection between detectors, similar to a gap between monitors in a dual monitor computer setup. While gaps can be narrowed by reducing bezel size or even placing detector panels in contact or nearly in contact with one another, images may still have missing data or other artifacts where different panels meet one another. A gap between detector areas can be, for example, 5 mm or about 5 mm, 10 mm or about 10 mm, 15 mm or about 15 mm, 20 mm or about 20 mm, or any number between these numbers, or more or less, depending upon the implementation. The gap can arise from the physical structure of the detector panels and the spacing required for mounting and positioning. Missing data in a gap region can significantly complicate image reconstruction.

Curved detectors can provide advantages over flat panel configurations. For example, a curved detector can be configured with an arc geometry where the center of the arc can be positioned at the focal spot of the X-ray source, such that each point on the detector is equidistant from the X-ray source. In flat panel detector configurations, detector elements positioned toward the outer edges of the detector can be located at greater distances from the X-ray source as compared with more central detector elements. X-ray flux can follow an inverse square law, resulting in significantly reduced flux and reduced signal-to-noise ratios near the outer edges of the detector(s).

The equidistant positioning of detector elements can provide improved, and consistent, signal-to-noise ratios across the detector surface, which can improve reconstruction and make it easier to account for scattering and other effects. A large, curved detector can provide various advantages. Detector elements toward the edge of the detector are closer to the x-ray source than they would be for a linear detector array, resulting in a higher signal-to-noise ratio (SNR) for these pixels. There is no dead space between panels as there would be when building an array from multiple detectors. Further, the curved detected can more easily be fit between the patient couch and the proton nozzle, enabling a large (e.g., about 50 cm) reconstructed CBCT field of view with a short-arc scan.

Curved panel detectors can eliminate or reduce gaps in the active detection area that may be present in tiled flat panel configurations. A monolithic curved detector can provide continuous detection coverage across an extended field of view, which can improve reconstruction quality. It will be appreciated that, in some implementations, curved detectors can be used in a tiled configuration. A tiled, curved detector configuration can achieve some advantages, such as maintaining a uniform distance from the X-ray source, while other challenges, such as gaps in data, can remain. The specific detectors chosen can depend upon availability, cost, desired image quality, etc.

A CBCT imaging system, as described herein, can incorporate scatter rejection and/or correction systems to address x-ray scatter that can degrade image quality in cone beam imaging applications. X-ray scatter may be more pronounced in CBCT systems relative to helical CT systems due to the larger cone angles used in CBCT imaging. Scatter rejection and correction systems can include hardware-based scatter rejection components, software-based scatter correction methods, or both.

Scatter rejection grids can be attached to each detector to reduce the amount of scattered X-rays that reach the detector surface. In some implementations, a scatter detection grid is focused on the X-ray source focal spot to selectively allow X-rays originating from the X-ray source to pass through while limiting the absorption of scattered X-rays. The grid can be an array of focused plates or channels that allow X-rays from the source to pass while blocking others. However, it will be appreciated that rejection grids are not perfect, and some scattered X-rays may still reach the detector.

Scatter rejection grids can be implemented in different configurations depending upon the specific application and system design. For example, scatter rejection grids can be one-dimensional (1D) or two-dimensional (2D). A 1D grid can include an array of plates arranged to provide scatter rejection along one dimension of the detector surface. The plates can be oriented to allow x-rays traveling along direct paths from the x-ray source to pass through, while blocking scattered x-rays traveling at different angles. 2D grids can include an array of focused channels that provide scatter rejection along both dimensions of the detector surface. The focused channels can be arranged in a grid pattern across the detector surface, with each channel oriented to reject scattered x-rays and prevent them from reaching the detector.

In some implementations, software-based scatter approaches are used additionally or alternatively. For example, Monte Carlo calculations can be used to estimate the scatter contribution in each projection image. The Monte Carlo scatter estimation can use an initial reconstructed image that has been generated without scatter correction as input for the scatter modeling process. The initial image can provide information about the patient's anatomy and tissue distribution that can be used to model the scattering behavior of X-rays as they pass through the patient.

The Monte Carlo calculations can simulate the interaction of X-rays with patient tissue based on anatomical information from the initial reconstructed image. For example, the simulation can model various scattering mechanisms, such as Compton scattering and coherent scattering, that can occur as x-rays traverse different tissue types. The Monte Carlo simulation can generate. Estimates of the scattered X-ray distribution that reaches each detector element across a range of projection angles.

The scatter estimates generated by the Monte Carlo calculations can be used to correct images for final image reconstruction. The scatter correction process can subtract the estimated scatter contribution from each projection image to obtain corrected projection data that more accurately represents the primary x-ray transmission through the patient, resulting in improved HU accuracy.

While Monte Carlo simulations can be effective, they can also be computationally demanding. Accordingly, it can be desirable to use other approaches that are faster, more computationally efficient, etc. In some implementations, a machine learning model is used for scatter correction. For example, a machine learning model can be trained to correct scatter by training the model using raw images that include scattering and corrected images with scattering removed (for example, using Monte Carlo simulations). The model can be trained to modify the raw images to produce images similar to the de-scattered images. Machine learning methods can be particularly effective for scatter correction, as scatter tends to be smooth and predictable.

While the above-discussed approaches can alleviate many issues with CBCT imaging, there is still the problem of missing data due to gaps between sensors, incomplete rotations, and so forth. Missing data presents a significant challenge as reconstruction has to rely on nearby data to fill in the missing data, which can lead to significant inaccuracies in the reconstructed image. For example, in the case of a gap, the gap can lead to missing data along an entire vertical or horizontal line, making reconstruction challenging. One way of viewing the projection data that has been measured is the sinogram, which plots the angle each projection ray makes with respect to the tomograph's coordinate system versus its distance from the axis of rotation. Because all angular information for radii near the axis of rotation is missing, reconstructed images can have severe artifacts.

In some implementations, a CBCT imaging system implements a wobbled scan orbit, which can effectively “spread” the missing information. The X-ray source and detector assembly can translate relative to the patient during the scan, such that missing information is spread around a variety of radii rather than concentrated in a single area. In some implementations, the X-ray source and detector translate. In other implementations, the patient is moved during the scan. For example, a patient couch can be motorized such that it can be translated during a scan. The translation can be carried out in an oscillating manner (e.g., a sinusoidal pattern of translation left and right).

When using translation of the patient couch, in order to match the wobbling behavior of the CBCT ring, where translation can be perpendicular to the principal line between the source and detector center, the patient couch can translate in both the x and y directions (assuming z is in the same direction as the axis of rotation of the CBCT system). This can be accomplished in a variety of ways, such as a single rail system that translates along a single direction forming an angle between the x and y axes, via multiple (e.g., two) rails that can be operated independently, or using other movement systems that enable movement in both x and y directions, either together or independently. For example, the patient couch can move along one rail (or similar system) that can be rotated, for example, in the x-z plane, where y is orthogonal to x and y and oriented vertically.

There can be any suitable number of oscillations per scan. For example, there can be half of an oscillation, a full oscillation, two oscillations, three oscillations, four oscillations, etc. In some implementations, a single oscillation is used during a scan. The number of oscillations can be limited in order to avoid causing the patient to experience motion sickness or to be jostled in the case that the patient couch is oscillating. Mechanical limitations (e.g., turning points, mechanical slop) can limit the speed of oscillation. It is important that the position of the patient be well-known and well-controlled during imaging in order to avoid reconstruction errors, blur, and so forth that can occur if there is motion that is unaccounted for or if there are positioning errors.

The wobbling motion can be characterized by a frequency and amplitude, with larger amplitudes spreading missing information over a larger area. The wobble distance can be, for example, 1 cm, 10 cm, 20 cm, or any other desired distance. In practice, the distance can be limited by scan capture time, which may be on the order of seconds to a few minutes, and the speed with which the detector and source (or patient couch) can be translated accurately and reliably. In general, the oscillation amplitude can be of the same distance as a detector dead zone. For example, if a dead zone is 16 mm (e.g., a 16 mm gap between detectors), a movement of 16 mm may be desirable. Such movements can distribute missing data through a larger portion of an image, enabling improved reconstruction.

The wobbling motion can be accomplished through various mechanical configurations, depending upon whether the X-ray source and detector are moved or the patient couch is moved. In implementations where the X-ray source and detector assembly are translated, the system can include a translation mechanism comprising one or more servo motors or stepper motors coupled to linear actuators or ball screw assemblies. The motors can be mounted to a support frame or gantry structure and connected to the detector assembly and x-ray source through mechanical linkages such as linear rails, guide rods, precision slides, etc, which can enable smooth, accurate translation along the desired axis. Position encoders can be integrated with the translation mechanism to provide precise feedback on the position of the imaging components during the wobble motion.

In implementations where the patient couch is translated, the couch can be equipped with a motorized translation system. The couch translation mechanism can include servo motors or stepper motors connected to linear actuators, ball screws, or rack-and-pinion systems that enable precise movement of the couch. The couch can be mounted on linear bearings or rails that allow smooth translation while maintaining stability and patient safety. The translation mechanism can be integrated with the imaging system's control electronics to synchronize the couch movement with the rotation of the X-ray source and detector. The system can include safety features such as limit switches, emergency stops, or patient restraints to ensure patient safety or limit patient movement during the wobble motion.

The mechanical systems for translation can be designed to provide precise, repeatable motion with little vibration or mechanical backlash that could affect image quality. In some implementations, motors are radiation-hardened or placed outside the primary radiation field to avoid interference with imaging or damage from radiation exposure. Control systems can include feedback loops using position encoders, accelerometers, or other sensors to ensure accurate positioning. The translation system can be programmed to execute various motion patterns, such as sinusoidal oscillation, circular wobble patterns, or other trajectories. The above-described examples are not limiting, and it will be appreciated that different rails, pins, motors, etc., can be used with the couch, the imaging system, or both to achieve reliable movements and motion tracking. While various sensors can be used for motion tracking, it will be appreciated that

Wobble can be readily accounted for during image reconstruction, as the translation is known, and pixels can readily be shifted to account for the wobble. For example, it can be readily determined from the wobble motion that a pixel value is shifted 15 mm to the right in an image, and the pixel value can be shifted to the left by 15 mm to account for the translation-induced shift.

The CBCT imaging system, as described herein, can be used for treatment planning. For example, each time a patient is to be treated, the patient can be positioned on a patient couch, and an image can be captured using the CBCT imaging system. The image can be used to more precisely position the patient or to steer the proton beam or control proton energy for depth control, thereby helping to ensure that treatment is delivered to the desired area and limiting exposure to nearby tissue and organs at risk.

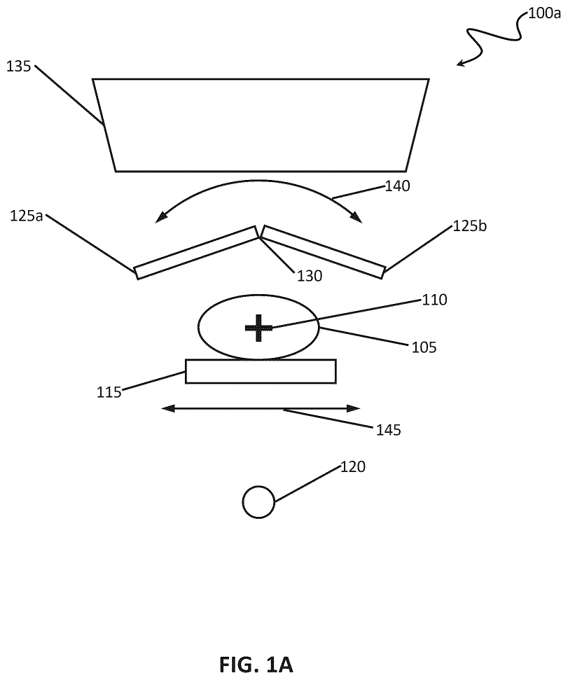

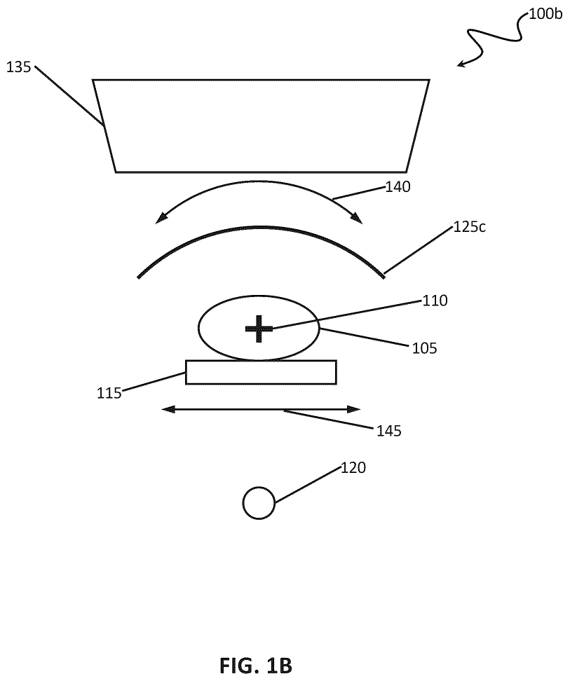

FIGS. 1A and 1B illustrate a top-down view of an imaging apparatus according to some implementations. A subject 105 can be imaged around an isocenter 110 (e.g., a treatment target, such as a tumor). The subject 105 can be placed on a patient couch 115 (also referred to herein as a couch). The patient couch 115 can be flat in some implementations, although other configurations are also possible. For example, the patient couch 115 can be formed as a chair in some implementations. In some implementations, the patient couch 115 can include various features, such as the ability to rotate, recline, and so forth, which can help to position the subject 105 in a preferable position for treatment, for example, in a position that avoids or limits exposure to organs at risk. A treatment gantry 135 can be used to provide protons to the treatment area. The treatment gantry 135 can include various features, such as electromagnetics, that can be used to steer, attenuate, or otherwise control a proton beam, which can be provided using a cyclotron, synchrotron, linear accelerator, etc.

The CBCT system can include an X-ray source 120 and one or more detectors. In configuration 100a of FIG. 1A, a first detector 125a and a second detector 125b are illustrated, with a gap or dead zone 130 between the detectors. The gap can be on the order of millimeters or centimeters and depends upon the specific detectors used and their placement. In configuration 100b of FIG. 1B, a single curved detector 125c is illustrated. The X-ray source 120 and detectors (125a and 125b in FIG. 1A, detector 125c in FIG. 1B) can be configured for rotation around the patient, as shown by arrow 140. The patient couch 115 can translate along the direction indicated by arrow 145 in some implementations. Additionally or alternatively, the X-ray source 120 and detector(s) can be configured to translate along the direction indicated by arrow 145.

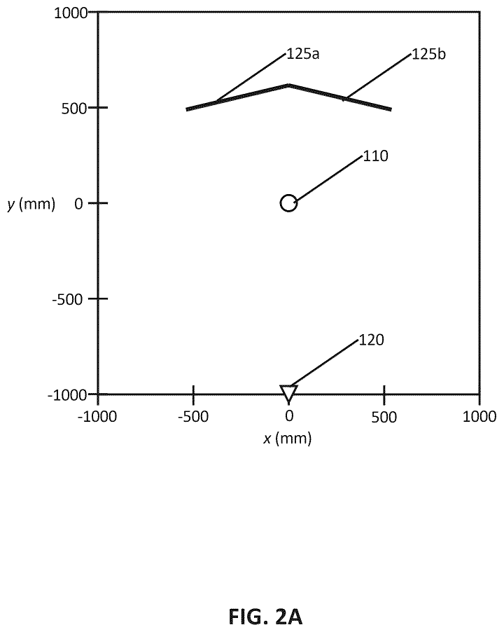

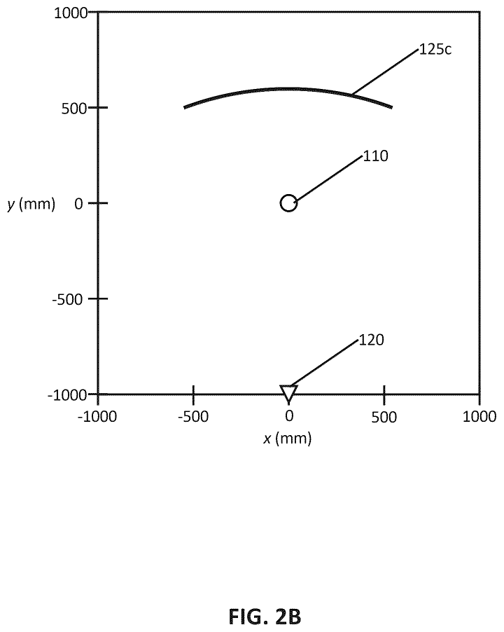

FIGS. 2A and 2B illustrate example geometries of systems according to FIGS. 1A and 1B, respectively. The coordinate axis can be centered around the isocenter 110, with the x-ray source 120 positioned in the center and away from the detector(s) (125a and 125b in FIGS. 2A, 125c in FIG. 2B). The detector and source can be configured to rotate around the isocenter 110. In some configurations, the x-ray source and detector can translate along the x-axis, or a patient (not shown) can translate along the x-axis, for example, on a motorized couch.

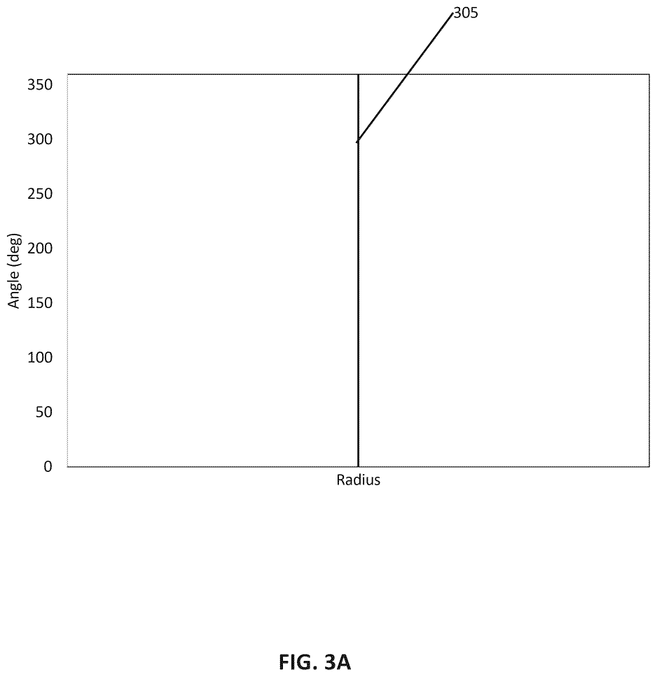

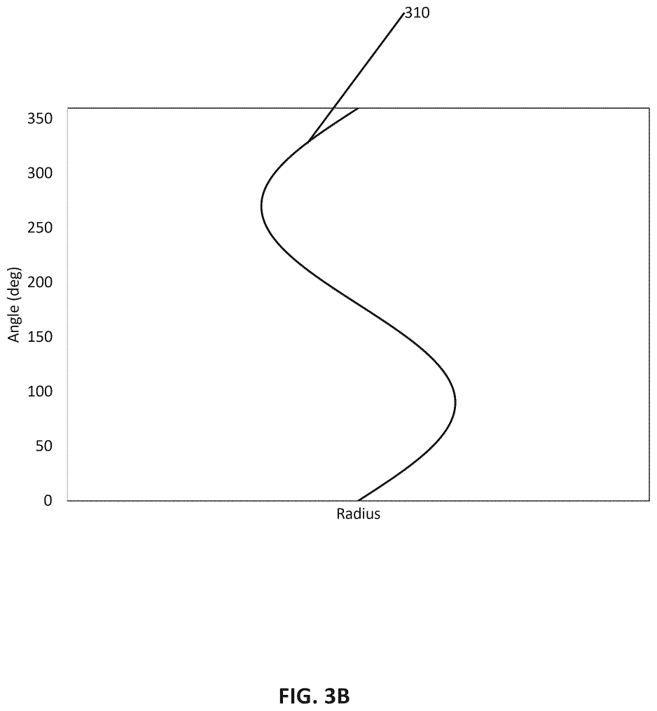

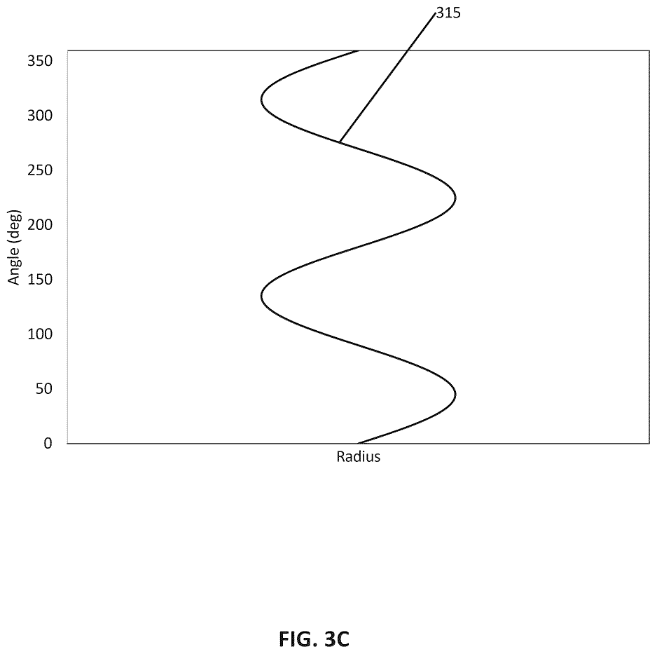

FIGS. 3A-3C illustrate example sinograms according to some implementations. FIG. 3A illustrates a configuration in which a two-detector array is used and there is a gap or dead zone between the detectors, which is reflected as the linear missing data 305. FIG. 3A illustrates missing data in the zero wobble (zero translation) case. FIGS. 3B and 3C represent sinograms with missing data 310 and 315, respectively. As shown in FIGS. 3B and 3C, the missing data is spread across multiple radii, rather than concentrated at zero. FIGS. 3B and 3C differ in the oscillatory frequency, with FIG. 3B illustrating one oscillation and FIG. 3C illustrating two oscillations.

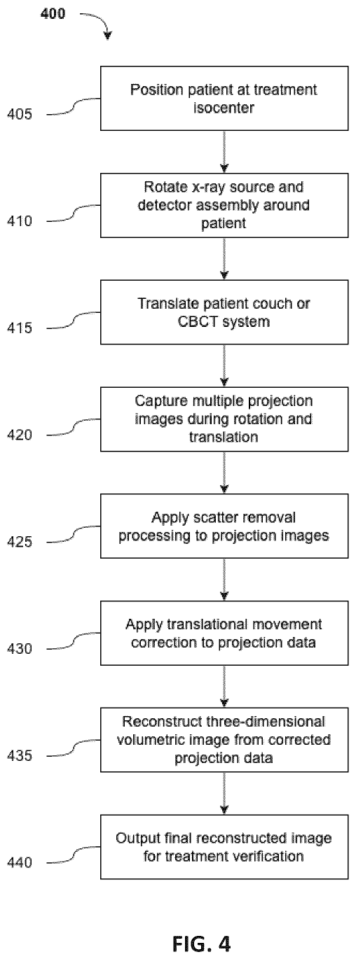

FIG. 4 illustrates a flowchart for a cone beam computed tomography imaging method 400 according to some implementations. The process of FIG. 4 can be used for proton therapy applications. At operation 405, a patient is positioned at a treatment isocenter. At operation 410, an X-ray source and detector assembly are rotated around the patient. At operation 415, either the patient couch or the CBCT system is translated to implement a wobbled scan orbit. As described herein, in some implementations, one or more sensors are used to monitor the position of the x-ray source and detector assembly, the patient couch, or both. At operation 420, projection images are captured during the simultaneous rotation and translation movements.

The captured projection data can undergo processing at operation 425, where scatter removal processing is applied to the projection images to reduce X-ray scatter contamination. At operation 430, a system can apply translational movement correction to the projection data to account for the wobbling motion introduced in operation 415. In some implementations, it can be preferable to apply translational movement correction before scatter correction, although it will be appreciated that this is not necessary. At operation 435, the system can reconstruct a three-dimensional volumetric image using the corrected projection data using a reconstruction algorithm. Various reconstruction algorithms can be used and can include, for example, analytical algorithms such as Feldkamp-Davis-Kress, Katsevich, or other filtered back-projection-type algorithms, or an iterative algorithm such as the algebraic reconstruction technique, simultaneous algebraic reconstruction technique, statistical iterative reconstruction, or sparsity-regularized iterative algorithms. At operation 440, the system can output the final reconstructed image for treatment verification purposes.

### EXAMPLES

Implementation 1. A cone beam computed tomography system for proton therapy, comprising: an x-ray source configured to generate x-rays and positioned to rotate around a patient at a treatment isocenter; a detector assembly positioned opposite the x-ray source and configured to receive x-rays that have passed through the patient, the detector assembly comprising at least one x-ray detector; a rotation mechanism configured to rotate the x-ray source and the detector assembly around the patient while maintaining their relative positions; a translation mechanism configured to translate at least one of the x-ray source and the detector assembly or a patient couch during rotation to implement a wobbled scan orbit; and a scatter rejection grid attached to the detector assembly and focused on the x-ray source to reduce scattered x-rays reaching the detector assembly.

Implementation 2. The cone beam computed tomography system of implementation 1, wherein the detector assembly comprises two flat-panel x-ray detectors arranged in a tiled configuration with a gap between the detectors.

Implementation 3. The cone beam computed tomography system of implementation 2, wherein the gap between the detectors is approximately 16 millimeters.

Implementation 4. The cone beam computed tomography system of implementation 1, wherein the detector assembly comprises a curved detector panel configured with an arc geometry centered at a focal spot of the x-ray source.

Implementation 5. The cone beam computed tomography system of implementation 1, wherein the detector assembly comprises two detectors, and wherein the translation mechanism is configured to translate the x-ray source and the detector assembly along a linear path having an amplitude of at least a gap size between the two detectors.

Implementation 6. The cone beam computed tomography system of implementation 5, wherein the translation mechanism is configured to perform a wobbling motion at a rate of rotation of the rotation mechanism.

Implementation 7. The cone beam computed tomography system of implementation 1, wherein the translation mechanism comprises linear rails and one or more motors configured to translate the patient couch in an oscillating pattern during the scan.

Implementation 8. The cone beam computed tomography system of implementation 1, wherein the scatter rejection grid comprises a two-dimensional array of focused channels oriented to reject scattered x-rays along both dimensions of the detector assembly.

Implementation 9. The cone beam computed tomography system of implementation 1, further comprising a scatter correction system configured to estimate scatter contribution using Monte Carlo calculations based on an initial reconstructed image.

Implementation 10. The cone beam computed tomography system of implementation 9, wherein the scatter correction system further comprises a machine learning model trained to generate scatter estimates.

Implementation 11. A method of cone beam computed tomography imaging for proton therapy, comprising: positioning a patient at a treatment isocenter between an x-ray source and a detector assembly; rotating the x-ray source and the detector assembly around the patient; translating at least one of the x-ray source and the detector assembly or a patient couch during the rotating to implement a wobbled scan orbit that distributes missing projection data across multiple radii and angles; capturing multiple projection images during the rotating and translating; applying scatter correction processing to the projection images to reduce x-ray scatter contamination; correcting the projection images for translational movement; and reconstructing a three-dimensional volumetric image from the corrected projection images.

Implementation 12. The method of implementation 11, wherein the detector assembly comprises two flat-panel x-ray detectors arranged in a tiled configuration with a gap between the detectors.

Implementation 13. The method of implementation 12, wherein the translating distributes missing projection data from the gap across multiple radii and angles in a sinogram representation.

Implementation 14. The method of implementation 12, wherein the translating comprises moving the x-ray source and the detector assembly along a linear path having an amplitude of at least the gap.

Implementation 15. The method of implementation 14, wherein the translating is performed at a rate or at twice the rate of the rotating.

Implementation 16. The method of implementation 11, wherein the translating comprises oscillating the patient couch in a sinusoidal pattern during the rotating.

Implementation 17. The method of implementation 11, wherein the applying scatter correction processing comprises using Monte Carlo calculations to estimate scatter contribution based on an initial reconstructed image without scatter correction.

Implementation 18. The method of implementation 17, wherein the applying scatter correction processing further comprises using a machine learning model trained to generate scatter estimates more rapidly than the Monte Carlo calculations.

Implementation 19. The method of implementation 11, wherein the reconstructing comprises using iterative reconstruction methods that account for missing projection data.

Implementation 20. The method of implementation 11, further comprising using the three-dimensional volumetric image for patient positioning verification prior to proton therapy treatment delivery.

### Computer System

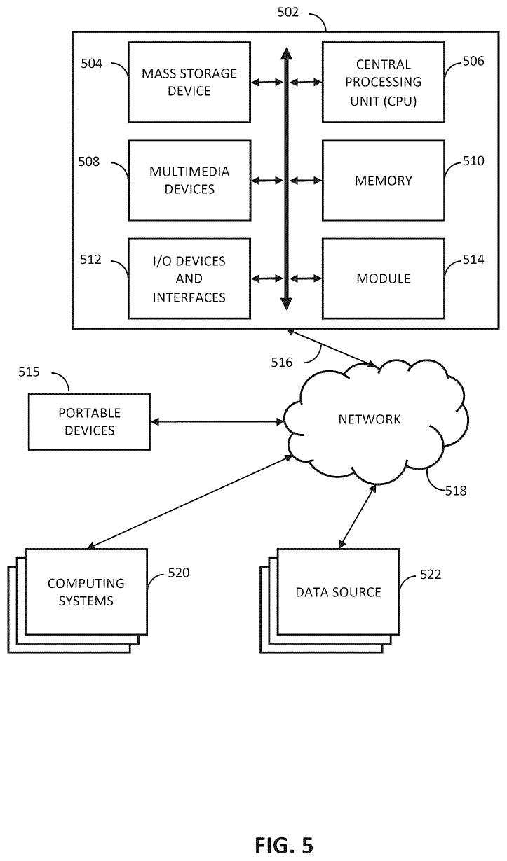

FIG. 5 is a block diagram 500 depicting an embodiment of a computer hardware system 502 configured to run software for implementing one or more of the systems and methods described herein. The example computer system 502 is in communication with one or more computing systems 520 and/or one or more data sources 522 via one or more networks 518. While FIG. 5 illustrates an embodiment of a computing system 502, it is recognized that the functionality provided for in the components and modules of computer system 502 may be combined into fewer components and modules, or further separated into additional components and modules.

The computer system 502 can comprise a module 514 that carries out the functions, methods, acts, and/or processes described herein. The module 514 is executed on the computer system 502 by a central processing unit 506, discussed further below.

In general, the word “module,” as used herein, refers to logic embodied in hardware or firmware or to a collection of software instructions, having entry and exit points. Modules are written in a programming language, such as Java, C, C++, Python, or the like. Software modules may be compiled or linked into an executable program, installed in a dynamic link library, or may be written in an interpreted language such as BASIC, PERL, LUA, or PYTHON. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer-readable medium or implemented in whole or in part within specially designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 502 includes one or more processing units (CPU) 506, which may comprise a microprocessor. The computer system 502 further includes a physical memory 510, such as random-access memory (RAM) for temporary storage of information, a read only memory (ROM) for permanent storage of information, and a mass storage device 504, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 502 are connected to the computer using a standards-based bus system. The bus system can be implemented using various protocols, such as Peripheral Component Interconnect (PCI), Micro Channel, SCSI, Industrial Standard Architecture (ISA), and Extended ISA (EISA) architectures.

The computer system 502 includes one or more input/output (I/O) devices and interfaces 512, such as a keyboard, mouse, touch pad, and printer. The I/O devices and interfaces 512 can include one or more display devices, such as a monitor, which allows the visual presentation of data to a user. More particularly, a display device provides for the presentation of GUIs as application software data, and multimedia presentations, for example. The I/O devices and interfaces 512 can also provide a communications interface to various external devices. The computer system 502 may comprise one or more multimedia devices 508, such as speakers, video cards, graphics accelerators, and microphones, for example.

The computer system 502 may run on a variety of computing devices, such as a server, a Windows server, a Structured Query Language server, a Unix Server, a personal computer, a laptop computer, and so forth. In other embodiments, the computer system 502 may run on a cluster computer system, a mainframe computer system, and/or other computing system suitable for controlling and/or communicating with large databases, performing high-volume transaction processing, and generating reports from large databases. The computing system 502 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 502 illustrated in FIG. 5 is coupled to a network 518, such as a LAN, WAN, or the Internet, via a communication link 516 (wired, wireless, or a combination thereof). Network 518 communicates with various computing devices and/or other electronic devices, such as portable devices 515. Network 518 is communicating with one or more computing systems 520 and one or more data sources 522. The module 514 may access or may be accessed by computing systems 520 and/or data sources 522 through a web-enabled user access point. Connections may be a direct physical connection, a virtual connection, or another connection type. The web-enabled user access point may comprise a browser module that uses text, graphics, audio, video, and other media to present data and to allow interaction with data via the network 518.

Access to the module 514 of the computer system 502 by computing systems 520 and/or by data sources 522 may be through a web-enabled user access point such as the computing systems'520 or data source's 522 personal computer, cellular phone, smartphone, laptop, tablet computer, e-reader device, audio player, or another device capable of connecting to the network 518. Such a device may have a browser module that is implemented as a module that uses text, graphics, audio, video, and other media to present data and to allow interaction with data via the network 518.

The output module may be implemented as a combination of an all-points addressable display, such as a cathode ray tube (CRT), a liquid crystal display (LCD), a plasma display, an organic light-emitting display (OLED display), or other types and/or combinations of displays. The output module may be implemented to communicate with interfaces 512, and can also include software with the appropriate interfaces that allow a user to access data through the use of stylized screen elements, such as menus, windows, dialogue boxes, toolbars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly, such as through a system terminal connected to the score generator without communications over the Internet, a WAN, or LAN, or a similar network.

In some implementations, the system 502 may comprise a physical or logical connection established between a remote microprocessor and a mainframe host computer for the express purpose of uploading, downloading, or viewing interactive data and databases online in real time. The remote microprocessor may be operated by an entity operating the computer system 502, including the client server systems or the main server system, and/or may be operated by one or more of the data sources 522 and/or one or more of the computing systems 520. In some implementations, terminal emulation software may be used on the microprocessor for participating in the micro-mainframe link.

In some implementations, computing systems 520 that are internal to an entity operating the computer system 502 may access the module 514 internally as an application or process run by the CPU 506.

In some implementations, one or more features of the systems, methods, and devices described herein can utilize a URL and/or cookies, for example, for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requester. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, a domain name, a file extension, a host name, a query, a fragment, a scheme, a protocol identifier, a port number, a username, a password, a flag, an object, a resource name, and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a website). The cookie data can be encrypted to provide security for the creator. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 502 may include one or more internal and/or external data sources (for example, data sources 522). In some implementations, one or more of the data repositories and the data sources described above may be implemented using a relational database, such as DB2, Sybase, Oracle, CodeBase, and Microsoft® SQL Server, as well as other types of databases, such as a flat-file database, an entity relationship database, and object-oriented database, and/or a record-based database.

The computer system 502 may also access one or more databases 522. The databases 522 may be stored in a database or data repository. The computer system 502 may access the one or more databases 522 through a network 518 or may directly access the database or data repository through I/O devices and interfaces 512. The data repository storing the one or more databases 522 may reside within the computer system 502.

### Conclusion

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment or implementation.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or operations. Thus, such conditional language is not generally intended to imply that features, elements and/or operations are in any way required for one or more embodiments or that one or more embodiments necessarily include logic for deciding, with or without author input or prompting, whether these features, elements and/or operations are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlaps, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions, including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles, and the novel features disclosed herein.

What is claimed is:

1. A cone beam computed tomography system for proton therapy, comprising:
an X-ray source configured to generate X-rays and positioned to rotate around a patient at a treatment isocenter;
a detector assembly positioned opposite the x-ray source and configured to receive x-rays that have passed through the patient, the detector assembly comprising at least one x-ray detector;
a rotation mechanism configured to rotate the X-ray source and the detector assembly around the patient while maintaining their relative positions;
a translation mechanism configured to translate at least one of the x-ray source and the detector assembly or a patient couch during rotation to implement a wobbled scan orbit; and
a scatter rejection grid attached to the detector assembly and focused on the x-ray source to reduce scattered x-rays reaching the detector assembly.

2. The cone beam computed tomography system of claim 1, wherein the detector assembly comprises two flat-panel x-ray detectors arranged in a tiled configuration with a gap between the detectors.

3. The cone beam computed tomography system of claim 2, wherein the gap between the detectors is approximately 16 millimeters.

4. The cone beam computed tomography system of claim 1, wherein the detector assembly comprises a curved detector panel configured with an arc geometry centered at a focal spot of the x-ray source.

5. The cone beam computed tomography system of claim 1, wherein the detector assembly comprises two detectors, and wherein the translation mechanism is configured to translate the x-ray source and the detector assembly along a linear path having an amplitude of at least a gap size between the two detectors.

6. The cone beam computed tomography system of claim 5, wherein the translation mechanism is configured to perform a wobbling motion at a rate of rotation of the rotation mechanism.

7. The cone beam computed tomography system of claim 1, wherein the translation mechanism comprises linear rails and one or more motors configured to translate the patient couch in an oscillating pattern during the scan.

8. The cone beam computed tomography system of claim 1, wherein the scatter rejection grid comprises a two-dimensional array of focused channels oriented to reject scattered x-rays along both dimensions of the detector assembly.

9. The cone beam computed tomography system of claim 1, further comprising a scatter correction system configured to estimate scatter contribution using Monte Carlo calculations based on an initial reconstructed image.

10. The cone beam computed tomography system of claim 9, wherein the scatter correction system further comprises a machine learning model trained to generate scatter estimates.

11. A method of cone beam computed tomography imaging for proton therapy, comprising:
positioning a patient at a treatment isocenter between an X-ray source and a detector assembly;
rotating the X-ray source and the detector assembly around the patient;
translating at least one of the x-ray source and the detector assembly or a patient couch during the rotating to implement a wobbled scan orbit that distributes missing projection data across multiple radii and angles;
capturing multiple projection images during the rotating and translating;
applying scatter correction processing to the projection images to reduce X-ray scatter contamination;
correcting the projection images for translational movement; and
reconstructing a three-dimensional volumetric image from the corrected projection images.

12. The method of claim 11, wherein the detector assembly comprises two flat-panel x-ray detectors arranged in a tiled configuration with a gap between the detectors.

13. The method of claim 12, wherein the translating distributes missing projection data from the gap across multiple radii and angles in a sinogram representation.

14. The method of claim 12, wherein the translating comprises moving the x-ray source and the detector assembly along a linear path having an amplitude of at least the gap.

15. The method of claim 14, wherein the translating is performed at a rate or at twice the rate of the rotating.

16. The method of claim 11, wherein the translating comprises oscillating the patient couch in a sinusoidal pattern during the rotating.

17. The method of claim 11, wherein the applying scatter correction processing comprises using Monte Carlo calculations to estimate scatter contribution based on an initial reconstructed image without scatter correction.

18. The method of claim 17, wherein the applying scatter correction processing further comprises using a machine learning model trained to generate scatter estimates more rapidly than the Monte Carlo calculations.

19. The method of claim 11, wherein the reconstructing comprises using iterative reconstruction methods that account for missing projection data.

20. The method of claim 11, further comprising using the three-dimensional volumetric image for patient positioning verification prior to proton therapy treatment delivery.
